(19) United States
(12) Patent Application Publication
(10) Pub. No.: US 2013/0116586 A1
(43) Pub. Date: May 9, 2013

(54) USE OF DRONEDARONE OR A PHARMACEUTICALLY ACCEPTABLE SALT THEREOF FOR THE PREPARATION OF A DRUG FOR THE PREVENTION OF ATRIAL FIBRILLATION

(71) Applicants: Lydie BARET-CORMEL, Paris (FR); Paul Chew, Bridgewater, NJ (US); Richard Gurai, Bridgewater, NJ (US); Davide RADZIK, Paris (FR); Antonio Tataranni, Paris (FR); Estelle WEINLING, Paris (FR)

(72) Inventors: Lydie BARET-CORMEL, Paris (FR); Paul Chew, Bridgewater, NJ (US); Richard Gurai, Bridgewater, NJ (US); Davide RADZIK, Paris (FR); Antonio Tataranni, Paris (FR); Estelle WEINLING, Paris (FR)

(73) Assignee: SANOFI, Paris (FR)

(21) Appl. No.: 13/541,144

(22) Filed: Jul. 3, 2012

Related U.S. Application Data
(60) Provisional application No. 61/505,410, filed on Jul. 7, 2011.
(60) Provisional application No. 61/537,786, filed on Sep. 22, 2011.

(30) Foreign Application Priority Data
Sep. 22, 2011 (EP) 11306205.3
Nov. 14, 2011 (EP) 11306482.8
Dec. 16, 2011 (EP) 11306688.0
Jun. 1, 2012 (FR) 1255112

(51) Int. Cl. A61B 5/046; A61K 31/343; A61B 5/00

(52) U.S. Cl. CPC A61B 5/046; A61B 5/4839; A61K 31/343
USPC 600/518

(57) ABSTRACT

The present invention relates to the use of dronedarone or a pharmaceutically acceptable salt thereof for the preparation of a drug for the prevention of atrial fibrillation wherein said use comprises the following precaution of use steps:
a) initiating dronedarone administration in patients with atrial fibrillation or atrial flutter,
b) performing electrocardiograms serially.

The present invention relates to the use of dronedarone or a pharmaceutically acceptable salt thereof for the preparation of a drug for the prevention of atrial fibrillation, said use comprising precaution of use steps.

The present invention also relates to the management of the risk for patients developing permanent atrial fibrillation said patients being treated by dronedarone or a pharmaceutically acceptable salt thereof.

The present invention also relates to dronedarone or pharmaceutically acceptable salts thereof, for use in the prevention of cardiovascular hospitalization and/or of mortality in a patient in need thereof, wherein the patient does not have permanent atrial fibrillation.

The present invention also relates to methods of treating or preventing atrial fibrillation or atrial flutter in a patient in need thereof, wherein the patient does not have permanent atrial fibrillation.

2-n-Butyl-3-[4-(3-di-n-butylaminopropoxy)benzoyl]-5-methylsulphonamidobenzofuran, or dronedarone, and pharmaceutically acceptable salts thereof are described in European Patent EP 0 471 609 B1.

Dronedarone blocks potassium, sodium and calcium channels and also has anti-adrenergic properties.

Dronedarone is an anti-arrhythmic that is effective in maintaining sinus rhythm in patients presenting with atrial fibrillation or atrial flutter.

### LIST OF ABBREVIATIONS

AF/AFL atrial fibrillation or atrial flutter
ATHENA A placebo-controlled, double-blind, parallel arm Trial to assess the efficacy of dronedarone 400 mg bid for the prevention of cardiovascular Hospitalization or death from any cause in patiENts with Atrial fibrillation/atrial flutter
PALLAS Permanent Atrial fibriLLAtion Outcome Study Using Dronedarone on Top of Standard Therapy

Atrial fibrillation is the most common arrhythmia that requires treatment, and is responsible for substantial morbidity and expenditure of health care dollars in the US and throughout the world. (Go AS, et al., JAMA 2001; 285: 2370-2375). While rhythm control has not been demonstrated to provide a survival benefit, as compared with simply controlling the rate with AV nodal blocking, many patients desire maintenance of sinus rhythm to improve symptoms. (Roy D, et al., New Engl J Med 2008; 358:2667-2677). Although ablation has been established to be a good option in many cases, drug therapy remains first-line therapy and is desired in many cases. Drug therapy for maintenance has been limited in terms of both efficacy and safety. (Roy D, et al., New Engl J Med 2008; 358:2667-2677).

The use of benzofuran derivatives to reduce post-infarction mortality in patients having a reduced left ventricular function after myocardial infarction, without any rhythm disorder requiring an anti-arrhythmic treatment, is known from Patent Applications WO 98/40067 and WO 97/34597.

In WO2009/144550, dronedarone has been shown to reduce relevant clinical endpoints such as cardiovascular hospitalizations or death in patients with atrial fibrillation (AF) and additional risk factors and notably in patients with paroxysmal or persistent atrial fibrillation (AF) or atrial flutter (AFL), with a recent episode of AF/AFL and associated cardiovascular risk factors (i.e., age >70, hypertension, diabetes, prior cerebrovascular accident, left atrial diameter ≧50 mm or left ventricular ejection fraction [LVEF]<40%), who are in sinus rhythm or who will be cardioverted.

Dronedarone is a multichannel blocking antiarrhythmic drug for the treatment of patients with atrial fibrillation. In a dose-ranging study (Touboul P, et al., Eur Heart J2003 August; 24(16):1481-1487), and two large randomized placebo-controlled trials (EURIDIS and ADONIS), the rhythm-controlling efficacy of dronedarone has been demonstrated. (Singh B N, et al., N Engl J Med 2007; 357:987-999). In addition, it was shown in the ERATO trial that dronedarone has rate-controlling properties in patients with permanent atrial fibrillation. (Davy J M, et al., Am Heart J 2008; 156:52: e1-9). However, the ANDROMEDA trial, comparing dronedarone with placebo in patients hospitalized with symptomatic unstable heart failure and severe left ventricular systolic function, was terminated early due to increased mortality among the patients receiving dronedarone. (Kober L, et al., N Engl J Med 2008; 358:2678-2687). Of note, less than 40% of the patients in both arms of the study had atrial fibrillation at baseline.

ATHENA demonstrated that dronedarone reduced major clinical outcomes in patients with atrial fibrillation including a reduction in cardiovascular hospitalizations or cardiovascular mortality, and stroke. (Hohnloser S H, et al., N. Engl. J. Med. 2009; 360:668-678) (Connolly S J, et al., Circulation 2009; 120:1174-1180).

Despite the large number of individuals with permanent AF, this condition is understudied. The effects of various rate control strategies and specific pharmacological interventions have been addressed in just a few studies involving relatively small cohorts of patients with permanent AF.

Patients with permanent AF were excluded from the ATHENA, EURIDIS and ADONIS trials, but 296 of 2,327 patients randomized to placebo in the ATHENA trial (12.7%) and 178 of 2301 patients assigned to dronedarone (7.7%) displayed AF on all electrocardiograms (ECGs) recorded during the follow-up period. For the sake of hypothesis generation, these patients can be considered as having permanent AF. In a post-hoc exploratory analysis of this cohort, the primary study endpoint (time to first CV hospitalization or all-cause death) occurred in 95 patients in the placebo group and 46 in the dronedarone group (HR 0.742, 95% Cl 0.522-1.056; p=0.096). Considering first unplanned CV hospitalizations, 85 patients in the placebo group and 37 on dronedarone reached this secondary study endpoint (HR 0.67, 95% Cl 0.46-0.99; p=0.04). There was a similar trend toward reduction in major cardiovascular events (stroke, ACS, cardiovascular death). These observations from ATHENA supported testing the efficacy of dronedarone for prevention of major cardiovascular events in patients with permanent AF.

The PALLAS trial (Permanent Atrial fibriLLAtion outcome Study) using dronedarone on top of standard therapy was designed to evaluate the efficacy of dronedarone on major cardiovascular events in a patient population with permanent AF.

The primary efficacy objective of PALLAS was the reduction of:
1) major cardiovascular events (stroke, systemic arterial embolism, myocardial infarction or cardiovascular death), or
2) unplanned cardiovascular hospitalization or death from any cause.

A preliminary review of the unblinded data from the PALLAS study unexpectedly revealed a highly significant excess of events in the dronedarone group for both the co-primary outcomes as well as cardiovascular hospitalizations and heart failure events with no evidence of benefit in other secondary endpoints.

The subject of the invention is therefore the use of dronedarone or a pharmaceutically acceptable salt thereof for the preparation of a medicament for use in the prevention of atrial fibrillation wherein said use comprises the following precaution for use steps:
a) initiating dronedarone administration in patients with atrial fibrillation or atrial flutter,
b) performing electrocardiograms serially.

According to one embodiment, said use also comprises the following step:
c) if patient develops permanent atrial fibrillation or if symptomatic atrial fibrillation reoccurs, discontinuing dronedarone administration.

According to another embodiment, said use also comprises the following step:
c) if atrial fibrillation is detected, discontinuing dronedarone administration or cardioverting said patient.

Another subject of the invention is a method of risk management of patients developing permanent atrial fibrillation said patients being treated by dronedarone or a pharmaceutically acceptable salt thereof, which method comprises the following steps:
a) initiating dronedarone administration in patients with atrial fibrillation or atrial flutter,
b) performing electrocardiograms serially.

According to one embodiment, said method of risk management also comprises the following step:
c) if patient develops permanent atrial fibrillation or if symptomatic atrial fibrillation reoccurs, discontinuing dronedarone administration.

According to another embodiment, said method of risk management also comprises the following step:
c) if atrial fibrillation is detected, discontinuing dronedarone administration or cardioverting said patient.

It may be mentioned that electrocardiograms (ECGs) are performed to monitor the heart rhythm of the patient and thus to determine cardiac rhythm to see whether or not he is in atrial fibrillation. Consequently, step b) may be understood as a step of determining cardiac rhythm.

“Serially” means at least every six months, particularly at least once every three months.

Mention may be made that said prevention of atrial fibrillation is defined by at least one of the following:
cardiovascular hospitalization,
hospitalization for atrial fibrillation.
maintenance of sinus rhythm.

Mention may be made that said patient is defined by at least one of the following:
patient has non permanent atrial fibrillation or atrial flutter,
patient has paroxysmal or persistent atrial fibrillation or atrial flutter,
patient does not have permanent atrial fibrillation or flutter with additional cardiovascular risk factors,
patient does not have permanent atrial fibrillation nor a history of, or current heart failure,
patient does not have permanent atrial fibrillation nor symptomatic heart failure,
patient does not have permanent atrial fibrillation nor symptomatic heart failure with recent decompensation requiring hospitalization,
patient does not have permanent atrial fibrillation nor heart failure defined as NYHA class IV,
patient does not have permanent atrial fibrillation nor left ventricular systolic dysfunction.

The above mentioned permanent atrial fibrillation is defined by one or more of the following:
a) Atrial fibrillation duration greater than or equal to 6 months,
b) Duration unknown,
c) attempts to restore sinus rhythm no longer considered by the physician,
d) patients in atrial fibrillation who will not or cannot be cardioverted into normal sinus rhythm.

According to one embodiment, dronedarone or a pharmaceutically acceptable salt thereof is taken twice a day with a meal notably the morning and evening meals.

A subject of the invention is therefore a method of risk management of patients developing permanent atrial fibrillation said patients being treated by dronedarone or a pharmaceutically acceptable salt thereof, which method comprises the following steps:
a) initiating dronedarone administration in patients with atrial fibrillation or atrial flutter particularly patients with non-permanent AF, more particularly patients with paroxysmal or persistent AF,
b) performing electrocardiograms (ECGs) serially.

A subject of the invention is therefore a method of risk management of patients developing permanent atrial fibrillation said patients being treated by dronedarone or a pharmaceutically acceptable salt thereof, which method comprises the following steps:
a) initiating dronedarone administration in patients with atrial fibrillation or atrial flutter particularly patients with non-permanent AF, more particularly patients with paroxysmal or persistent AF,
b) performing electrocardiograms (ECGs) serially,
c) if patient develops permanent AF, discontinuing dronedarone administration.

A subject of the invention is therefore a method of risk management of patients developing permanent atrial fibrillation said patients being treated by dronedarone or a pharmaceutically acceptable salt thereof, which method comprises the following steps:
a) initiating dronedarone administration in patients with atrial fibrillation or atrial flutter particularly patients with non-permanent AF, more particularly patients with paroxysmal or persistent AF,
b) performing electrocardiograms (ECGs) serially,
c) if atrial fibrillation is detected, discontinuing dronedarone administration or cardioverting the patient.

A subject of the invention is therefore a method of risk management of patients developing permanent atrial fibrillation said patients being treated by dronedarone or a pharmaceutically acceptable salt thereof, which method comprises the following steps:
a) initiating dronedarone administration in patients with atrial fibrillation or atrial flutter particularly patients with non-permanent AF, more particularly patients with paroxysmal or persistent AF,
b) performing electrocardiograms (ECGs) serially,
c) if symptomatic atrial fibrillation reoccurs, discontinuing dronedarone administration.

It may be mentioned that electrocardiograms (ECGs) are performed to monitor the heart rhythm of the patient and thus to determine cardiac rhythm to see whether or not he is in atrial fibrillation. Consequently, step b) may be understood as a step of determining cardiac rhythm.

“Serially” means at least every six months, particularly at least once every three months.

According to one embodiment, the invention is a method of risk management of patients developing permanent atrial fibrillation said patients being treated by dronedarone or a pharmaceutically acceptable salt thereof, which method comprises the following steps:
a) initiating dronedarone administration in patients with atrial fibrillation or atrial flutter particularly patients with non-permanent AF, more particularly patients with a history of paroxysmal or persistent AF,
b) determine cardiac rhythm at least once every three months,
c) if atrial fibrillation is detected, discontinuing dronedarone administration or cardioverting said patient.

Another subject of the present invention is dronedarone or a pharmaceutically acceptable salt thereof for use in the treatment of atrial fibrillation or flutter in patients with paroxysmal or persistent AF, wherein said use involves the following steps:
a) initiating dronedarone administration in patients with atrial fibrillation or atrial flutter particularly patients with non-permanent AF, more particularly patients with paroxysmal or persistent AF,
b) performing ECGs serially,
c) if patient develops permanent AF, discontinuing treatment.

A subject of the present invention is also the use of dronedarone or a pharmaceutically acceptable salt thereof, for the preparation of a medicament for the prevention of cardiovascular hospitalizations and/or of mortality, wherein the patient does not have permanent atrial fibrillation or flutter.

A subject of the present invention is also the use of dronedarone or a pharmaceutically acceptable salt thereof, for the preparation of a medicament for the maintenance of sinus rhythm wherein the patient does not have permanent atrial fibrillation or flutter.

Another subject of the present invention is therefore the use of dronedarone or a pharmaceutically acceptable salt thereof, for the preparation of a medicament, for the prevention of cardiovascular hospitalizations and/or of mortality, wherein the patient does not have permanent atrial fibrillation or flutter with additional cardiovascular risk factors.

Another subject of the present invention is therefore the use of dronedarone or a pharmaceutically acceptable salt thereof, for the preparation of a medicament, for the maintenance of sinus rhythm, wherein the patient does not have permanent atrial fibrillation or flutter with additional cardiovascular risk factors.

A subject of the present invention is also the use of dronedarone or a pharmaceutically acceptable salt thereof, for the preparation of a medicament, taken twice a day with food notably the morning and evening meals, for the prevention of mortality and/or of cardiovascular hospitalizations, wherein said patient does not have permanent atrial fibrillation or flutter.

A subject of the present invention is also the use of dronedarone or a pharmaceutically acceptable salt thereof, for the preparation of a medicament, taken twice a day with food notably the morning and evening meals, for the prevention of mortality and/or of cardiovascular hospitalizations, wherein said patient does not have permanent atrial fibrillation or flutter with additional cardiovascular risk factors.

A subject of the present invention is also the use of dronedarone or a pharmaceutically acceptable salt thereof, for the preparation of a medicament, taken twice a day with food notably the morning and evening meals, for the maintenance of sinus rhythm, wherein said patient does not have permanent atrial fibrillation or flutter. In a particular embodiment, the patient does not have permanent AF with additional cardiovascular risk factors.

In an embodiment, the subject of the instant invention is the use of dronedarone or one of its pharmaceutically acceptable salts for the preparation of a medicament for use in the prevention of cardiovascular hospitalization and/or of mortality, wherein said patient does not have permanent AF and an history of, or current heart failure notably symptomatic heart failure or heart failure defined as NYHA class II, III or IV. In a particular embodiment, the patient does not have permanent AF with additional cardiovascular risk factors.

In an embodiment, the subject of the instant invention is the use of dronedarone or one of its pharmaceutically acceptable salts for the preparation of a medicament for use in the maintenance of sinus rhythm, wherein said patient does not have permanent AF and an history of, or current heart failure notably symptomatic heart failure or heart failure defined as NYHA class II, III or IV. In a particular embodiment, the patient does not have permanent AF with additional cardiovascular risk factors.

In an embodiment, the subject of the instant invention is the use of dronedarone or one of its pharmaceutically acceptable salts for the preparation of a medicament for the prevention of cardiovascular hospitalization and/or of mortality, wherein said patient does not have permanent AF and left ventricular systolic dysfunction. In a particular embodiment, the patient does not have permanent AF with additional cardiovascular risk factors.

In an embodiment, the subject of the instant invention is the use of dronedarone or one of its pharmaceutically acceptable salts for the preparation of a medicament for the maintenance of sinus rhythm, wherein said patient does not have permanent AF and left ventricular systolic dysfunction. In a particular embodiment, the patient does not have permanent AF with additional cardiovascular risk factors.

The present invention also relates to methods of treating patients with dronedarone, or a pharmaceutically acceptable salt thereof, wherein said patient does not have permanent atrial fibrillation. In a particular embodiment, the patient does not have permanent AF with additional cardiovascular risk factors.

In the context of the present invention, it is mentioned that a method of risk management or a method for managing a risk correspond to a precaution for use.

The present invention also relates to methods of preventing or treating atrial fibrillation in a patient with a history of paroxysmal or persistent atrial fibrillation or atrial flutter, comprising initiating dronedarone administration in said patient, performing electrocardiograms of said patient serially and discontinuing dronedarone administration if the patient develops permanent atrial fibrillation or if symptomatic atrial fibrillation reoccurs.

The present invention also relates to methods of reducing the risk of cardiovascular hospitalization for atrial fibrillation in a patient with a history of paroxysmal or persistent atrial fibrillation, comprising initiating dronedarone administration in said patient, performing electrocardiograms of said patient serially and discontinuing dronedarone administration if the patient develops permanent atrial fibrillation or if symptomatic atrial fibrillation reoccurs.

A subject of the instant invention also relates to dronedarone or one of its pharmaceutically acceptable salts for the treatment of atrial fibrillation or flutter in patients without permanent atrial fibrillation, a therapeutic amount of dronedarone or pharmaceutically acceptable salt thereof being administered. In a particular embodiment, the patient does not have permanent AF with additional cardiovascular risk factors

Another subject of the invention is performed by providing dronedarone or pharmaceutically acceptable salts thereof, wherein said dronedarone or pharmaceutically acceptable salts thereof is provided along with information indicating that as precaution for use, a method of managing the risk of patients developing permanent atrial fibrillation comprises the following steps:
a) initiating dronedarone administration in patients with paroxysmal atrial fibrillation or atrial flutter,
b) performing electrocardiograms (ECGs) serially,
c) if patient develops permanent AF or if symptomatic AF reoccurs, discontinuing dronedarone administration.

Another subject of the invention is performed by providing dronedarone or pharmaceutically acceptable salts thereof, wherein said dronedarone or pharmaceutically acceptable salts thereof is provided along with information indicating that as precaution for use, a method of managing the risk of patients developing permanent atrial fibrillation comprises the following steps:
a) initiating dronedarone administration in patients with atrial fibrillation or atrial flutter particularly patients with non-permanent AF, more particularly patients with a history of paroxysmal or persistent AF,
b) determine cardiac rhythm at least once every three months,
c) if atrial fibrillation is detected, discontinuing dronedarone administration or cardiovert.

Another subject of the invention is performed by providing dronedarone or pharmaceutically acceptable salts thereof, wherein said dronedarone or pharmaceutically acceptable salts thereof is provided along with information indicating that dronedarone or pharmaceutically acceptable salts thereof is contraindicated in patients with permanent atrial fibrillation. In a particular embodiment, the patient does not have permanent AF with additional cardiovascular risk factors

Another subject of the invention is performed by providing dronedarone or pharmaceutically acceptable salts thereof, wherein said dronedarone or pharmaceutically acceptable salts thereof is provided along with information indicating that dronedarone or pharmaceutically acceptable salts thereof should not be used in patients with permanent atrial fibrillation. In a particular embodiment, the patient does not have permanent AF with additional cardiovascular risk factors

In an embodiment of the invention, the information comprises printed matter that advises that as precaution for use of dronedarone or pharmaceutically acceptable salts, a method of managing the risk of patients developing permanent atrial fibrillation comprises the following steps:
a) initiating dronedarone administration in patients with atrial fibrillation or atrial flutter,
b) performing electrocardiograms (ECGs) serially,
c) if patient develops permanent AF or if symptomatic AF reoccurs, discontinuing dronedarone administration.

In an embodiment of the invention, the information comprises printed matter that advises that as precaution for use of dronedarone or pharmaceutically acceptable salts, a method of managing the risk of patients developing permanent atrial fibrillation comprises the following steps:
a) initiating dronedarone administration in patients with atrial fibrillation or atrial flutter particularly patients with non-permanent AF, more particularly patients with a history of paroxysmal or persistent AF,
b) determine cardiac rhythm at least once every three months,
c) if atrial fibrillation is detected, discontinuing dronedarone administration or cardiovert.

In an embodiment of the invention, the information comprises printed matter that advises that dronedarone or pharmaceutically acceptable salts thereof is indicated in patients with paroxysmal or persistent atrial fibrillation or atrial flutter with associated risk factors, wherein said patient does not have permanent AF. In a particular embodiment, the patient does not have permanent AF with additional cardiovascular risk factors. In another embodiment, the printed material is a label.

The invention also relates to a method of promoting the use of dronedarone or pharmaceutically acceptable salts thereof, the method comprising the step of conveying to a recipient at least one message selected from the group consisting of:

(a) dronedarone or pharmaceutically acceptable salts thereof should be prescribed to a patient who does not have permanent atrial fibrillation;
(b) dronedarone or pharmaceutically acceptable salts thereof is contraindicated in patients who have permanent atrial fibrillation;
(c) precaution for use of dronedarone or pharmaceutically acceptable salts, comprises the following steps:
a) initiating dronedarone administration in patients with atrial fibrillation or atrial flutter,
b) performing electrocardiograms (ECGs) serially,
c) if patient develops permanent AF or if symptomatic AF reoccurs, discontinuing dronedarone administration.

The invention also relates to a method of promoting the use of dronedarone or pharmaceutically acceptable salts thereof, the method comprising the step of conveying to a recipient at least one message selected from the group consisting of:

(a) dronedarone or pharmaceutically acceptable salts thereof should be prescribed to a patient who does not have permanent atrial fibrillation;
(b) dronedarone or pharmaceutically acceptable salts thereof is contraindicated in patients who have permanent atrial fibrillation;
(c) precaution for use of dronedarone or pharmaceutically acceptable salts, comprises the following steps:
a) initiating dronedarone administration in patients with atrial fibrillation or atrial flutter particularly patients with non-permanent AF, more particularly patients with a history of paroxysmal or persistent AF,
b) determine cardiac rhythm at least once every three months,
c) if atrial fibrillation is detected, discontinuing dronedarone administration or cardiovert.

The invention also relates to a package comprising dronedarone or pharmaceutically acceptable salts thereof and a label, said label comprising a printed statement which informs a prospective user that:

(a) dronedarone or pharmaceutically acceptable salts thereof is contraindicated in patients with permanent atrial fibrillation, and/or
(b) precaution for use of dronedarone or pharmaceutically acceptable salts, comprises the following steps:
a) initiating dronedarone administration in patients with atrial fibrillation or atrial flutter,
b) performing electrocardiograms (ECGs) serially,
c) if patient develops permanent AF or if symptomatic AF reoccurs, discontinuing dronedarone administration.

The invention also relates to a package comprising dronedarone or pharmaceutically acceptable salts thereof and a label, said label comprising a printed statement which informs a prospective user that:

(a) dronedarone or pharmaceutically acceptable salts thereof is contraindicated in patients with permanent atrial fibrillation, and/or
(b) precaution for use of dronedarone or pharmaceutically acceptable salts, comprises the following steps:
a) initiating dronedarone administration in patients with atrial fibrillation or atrial flutter particularly patients with non-permanent AF, more particularly patients with a history of paroxysmal or persistent AF,
b) determine cardiac rhythm at least once every three months,
c) if atrial fibrillation is detected, discontinuing dronedarone administration or cardiovert.

Another method of the invention comprises treating a patient with atrial fibrillation, said method comprising administrating to said patient a therapeutically effective amount of dronedarone, or a pharmaceutically acceptable salt thereof, wherein said patient does not have permanent atrial fibrillation. In a particular embodiment, the patient does not have permanent AF with additional cardiovascular risk factors.

Another method of the invention relates to transforming a patient with atrial fibrillation or flutter by decreasing the patient's risk of cardiovascular hospitalizations or mortality, comprising administrating to said patient a therapeutically effective amount of dronedarone, or a pharmaceutically acceptable salt thereof, wherein said patient does not have permanent atrial fibrillation. In a particular embodiment, the patient does not have permanent AF with additional cardiovascular risk factors.

A subject of the invention is a method of decreasing the risk of cardiovascular hospitalizations or mortality in a patient having a history of atrial fibrillation or atrial flutter, said method comprising administering dronedarone, or a pharmaceutically acceptable salt thereof, twice a day with a meal to a patient in need thereof, wherein said patient does not have permanent atrial fibrillation. In a particular embodiment, the patient does not have permanent AF with additional cardiovascular risk factors.

Mention may be made that

Said patients may additionally be defined by one or more of the following:
patients treated with dronedarone, and/or
patients having atrial fibrillation or atrial flutter, and/or
patients having non-permanent atrial fibrillation or atrial flutter, and/or
patients having paroxysmal or persistent atrial fibrillation or atrial flutter, and/or
patients having paroxysmal or persistent atrial fibrillation (AF) or atrial flutter (AFL), with a recent episode of AF/AFL and associated cardiovascular risk factors (i.e., age >70, hypertension, diabetes, prior cerebrovascular accident, left atrial diameter ≧50 mm or left ventricular ejection fraction [LVEF]<40%) and are in sinus rhythm or will be cardioverted, and/or
adult clinically stable patients with paroxysmal or persistent AF, and/or
adult clinically stable patients with paroxysmal or persistent AF after successful cardioversion, and/or
patients with paroxysmal or persistent atrial fibrillation when sinus rhythm has been obtained, and/or
patients with paroxysmal or persistent atrial fibrillation after successful cardioversion, and/or
patients having non-permanent atrial fibrillation or atrial flutter after successful cardioversion,
patients with a history of paroxysmal or persistent AF.
patients in sinus rhythm with a history of paroxysmal or persistent AF.

A sub-group of patients according to the invention may be patients as defined previously and having diabetes.

History of heart failure means that previous episodes of heart failure occurred.

NYHA means New York Heart Association.

Said “additional cardiovascular risk factors” presented in the “patients without permanent AF with additional cardiovascular risk factors” are defined by one or more of the following:
a) age greater or equal to 65 years old,
b) Coronary artery disease,
c) Prior stroke or TIA (confirmed by a neurologist),
d) An history of, or current heart failure, for example class I, II, III, IV NYHA heart failure,
e) Symptomatic heart failure,
f) NYHA class III heart failure
g) Left ventricular ejection fraction less than or equal to 0.40 measured by echocardiography within 3 months of randomization,
h) Left ventricular systolic dysfunction defined by left ventricular ejection fraction less than or equal to 0.40,
i) Left ventricular systolic dysfunction defined by impairment affecting the left side of the heart,
j) Peripheral arterial occlusive disease,
k) Aged 75 years or older with both hypertension and diabetes mellitus.

Said permanent atrial fibrillation is defined by one or more of the following:
a) Atrial fibrillation duration greater than or equal to 6 months,
b) Duration unknown,
c) attempts to restore sinus rhythm no longer considered by the physician,
d) patients in atrial fibrillation who will not or cannot be cardioverted into normal sinus rhythm.

The term “providing” includes selling, distributing, shipping, offering for sell, importing etc.

Mention may be made that “dronedarone for the treatment of” may be understood as “use of dronedarone for the preparation of a medicament for use in the treatment of” and vice-versa.

In terms of clinical study, the prevention of “major cardiovascular events” or of “unplanned cardiovascular hospitalization or death from any cause” constitute what are referred to as composite criteria or a combined endpoint.

The term “cardiovascular hospitalization” means a hospitalization which is caused by at least one of the following pathologies (Hohnloser et al., Journal of cardiovascular electrophysiology, January 2008, vol. 19, No. 1, pages 69-73):
relating to atherosclerosis,
myocardial infarction or unstable angina pectoris,
stable angina pectoris or atypical thoracic pain,
syncope,
transient ischemic event or cerebral stroke (except intracranial hemorrhage),
atrial fibrillation and other supraventricular rhythm disorders,
non-fatal cardiac arrest,
ventricular arrhythmia,
cardiovascular surgery, except heart transplant,
heart transplant,
implantation of a cardiac stimulator (pacemaker), of an implantable defibrillator (“ICD”) or of another cardiac device,
percutaneous coronary, cerebrovascular or peripheral intervention,
variations in arterial pressure (hypotension, hypertension, except syncope),
cardiovascular infection,
major bleeding/hemorrhage (requiring two or more blood cell pellets or any intracranial hemorrhage),
pulmonary embolism or deep vein thrombosis,
worsening of congestive heart failure including acute pulmonary oedema or dyspnoea from cardiac causes.

Consequently, the prevention of cardiovascular hospitalization may be understood as the prevention of hospitalization for at least one of the above mentioned pathologies.

Mention may be made of the prevention of hospitalization for atrial fibrillation.

Thus, a subject of the present invention is also the use of dronedarone or a pharmaceutically acceptable salt thereof, for the preparation of a medicament for the prevention of hospitalization for at least one of the above mentioned pathologies.

In one embodiment, a subject of the present invention is also the use of dronedarone or a pharmaceutically acceptable salt thereof, for the preparation of a medicament for the prevention of hospitalization for atrial fibrillation in patients with paroxysmal or persistent atrial fibrillation, in particular in patients in sinus rhythm with a history of paroxysmal or persistent atrial fibrillation, wherein said patients do not have permanent atrial fibrillation.

In another embodiment, a subject of the present invention is also the use of dronedarone or a pharmaceutically acceptable salt thereof, for the preparation of a medicament for the prevention of cardiovascular hospitalization for atrial fibrillation in patients with paroxysmal or persistent atrial fibrillation, in particular in patients in sinus rhythm with a history of paroxysmal or persistent atrial fibrillation, wherein said patients do not have symptomatic heart failure, notably symptomatic heart failure with recent decompensation requiring hospitalization or NYHA Class IV heart failure.

The term “mortality” or “death” are equivalent and cover mortality due to any cause, whether cardiovascular or non-cardiovascular or unknown.

The term “cardiovascular mortality” covers, in the context of the invention, mortality due to any cardiovascular causes (any death except those due to a non-cardiovascular cause), in particular death from an arrhythmic cause, also called arrhythmic death, and more particularly, sudden death from cardiovascular causes, also called sudden death or sudden cardiac death.

Cardiovascular mortality may be due for example to:
Aortic dissection/aneurysm
Cardiac tamponade
Cardiogenic shock
congestive heart failure
Death during a cardiovascular transcutaneous interventional procedure or cardiovascular surgical intervention
Hemorrhage (except cardiac tamponade)
Myocardial infarction or unstable angina (including complications of myocardial infarction, except arrhythmias)
Pulmonary or peripheral embolism
Stroke
Sudden cardiac death (e.g., unwitnessed death or documented asystole)
Ventricular arrhythmia, subclassified as torsades de pointes, ventricular extrasystole, ventricular fibrillation, ventricular tachycardia (non-sustained and sustained ventricular tachycardia), or other ventricular arrhythmia
Unknown cause

The term “sudden death” refers, in general, to death occurring within the hour or less than one hour after the appearance of new symptoms or unexpected death without warning.

The term “coronary disease” or “coronary heart disease” refers to:
1) Coronary artery disease: documented history of acute myocardial infarction and/or significant (≧70%) coronary artery stenosis and/or history of a revascularization procedure (percutaneous transluminal coronary angioplasty, stent implantation in a coronary artery, coronary artery bypass graft, etc) and/or a positive exercise test and/or positive nuclear scan of cardiac perfusion
2) Ischemic dilated cardiomyopathy: clinically significant left ventricular dilatation secondary to coronary artery disease

Among the patients, notably patients having a paroxysmal or persistent atrial fibrillation or atrial flutter, mention may also be made of patients also exhibiting at least one of associated cardiovascular risk factors, these “associated cardiovascular risk factors” are defined by at least one of the following:
age equal to or above 70, or even above 75,
hypertension,
diabetes,
history of cerebral stroke or of systemic embolism, i.e. prior cerebrovascular accident,
left atrial diameter greater than or equal to 50 mm,
left ventricular ejection fraction less than 40.

It may be understood that “prevention of cardiovascular hospitalization and/or mortality” results in the “reduction of the risk of cardiovascular hospitalization and or mortality” or in the “reduction of the need of cardiovascular hospitalization and or mortality”.

It may be understood that “prevention of hospitalization for atrial fibrillation” results in the “reduction of the risk of hospitalization for atrial fibrillation” or in the “reduction of the need of hospitalization for atrial fibrillation”.

Dronedarone and pharmaceutically acceptable salts thereof may be given once or twice a day with food for example with the morning and evening meals.

For their therapeutic use, dronedarone and pharmaceutically acceptable salts thereof are generally introduced into pharmaceutical compositions.

These pharmaceutical compositions contain an effective dose of dronedarone or of a pharmaceutically acceptable salt thereof, and also at least one pharmaceutically acceptable excipient.

Said pharmaceutical composition may be given once or twice a day with food.

The dose of dronedarone administered per day, orally, may reach 800 mg, taken in one or more intakes, for example one or two.

More specifically, the dose of dronedarone administered may be taken with food.

More specifically, the dose of dronedarone administered per day, orally, may reach 800 mg, taken in two intakes with a meal.

The dose of dronedarone administered per day, orally may be taken at a rate of twice a day with a meal for example with the morning and the evening meal.

More specifically, the two intakes may comprise same quantity of dronedarone.

There may be specific cases where higher or lower dosages are appropriate; such dosages do not depart from the context of the invention. According to the usual practice, the dosage appropriate for each patient is determined by the physician according to the method of administration, the weight, the pathology, the body surface, the cardiac output and the response of said patient.

Said excipients are chosen according to the pharmaceutical form and the method of administration desired, from the usual excipients which are known to those skilled in the art.

In said pharmaceutical compositions for oral, sublingual, subcutaneous, intramuscular, intravenous, topical, local, intratracheal, intranasal, transdermal or rectal administration, dronedarone, or the salt thereof, can be administered in unit administration form, as a mixture with conventional pharmaceutical excipients, to animals and to humans in the cases mentioned above.

The suitable unit administration forms comprise forms for oral administration, such as tablets, soft or hard gel capsules, powders, granules and oral solutions or suspensions, sublingual, buccal, intratracheal, intraocular or intranasal administration forms, forms for administration by inhalation, topical, transdermal, subcutaneous, intramuscular or intravenous administration forms, rectal administration forms, and implants. For topical application, dronedarone and pharmaceutically acceptable salts thereof can be used in creams, gels, ointments or lotions.

By way of example, a unit administration form of dronedarone or a pharmaceutically acceptable salt thereof, in tablet form, may correspond to one of the following examples:

| Ingredients | mg | % |
|---|---|---|
| Dronedarone hydrochloride | 426 | 65.5 |
| (corresponding to 400 mg of base) | | |
| Methylhydroxypropylcellulose | 21.1 | 3.25 |
| Lactose monohydrate | 46.55 | 7.2 |
| Maize starch | 45.5 | 7 |
| Polyvinylpyrrolidone | 65 | 10 |
| Poloxamer 407 | 40 | 6.15 |
| Anhydrous colloidal silica | 2.6 | 0.4 |
| Magnesium stearate | 3.25 | 0.5 |
| | 650 | 100 |
| Dronedarone hydrochloride | 426 | 65.5 |
| (corresponding to 400 mg of base) | | |
| Microcrystalline cellulose | 65 | 10 |
| Anhydrous colloidal silica | 2.6 | 0.4 |
| Anhydrous lactose | 42.65 | 6.6 |
| Polyvinylpyrrolidone | 13 | 2 |
| Poloxamer 407 | 40 | 6.15 |
| Macrogol 6000 | 57.5 | 8.85 |
| Magnesium stearate | 3.25 | 0.5 |
| | 650 | 100 |

| Ingredients | mg |
|---|---|
| Dronedarone hydrochloride | 426 |
| (corresponding to 400 mg of base) | |
| Microcrystalline cellulose | 26 |
| Maize starch | 45.5 |
| Polyvinylpyrrolidone | 65 |
| Poloxamer 407 | 40 |
| Anhydrous colloidal silica | 3.25 |
| Magnesium stearate | 3.25 |
| Lactose monohydrate | 41.65 |
| | 650 |
| Dronedarone hydrochloride | 213 |
| (corresponding to 200 mg of base) | |
| Microcrystalline cellulose | 13 |
| Maize starch | 22.75 |
| Polyvinylpyrrolidone | 32.5 |
| Poloxamer 407 | 20 |
| Anhydrous colloidal silica | 1.3 |
| Magnesium stearate | 1.625 |
| Lactose monohydrate | 20.825 |
| | 325 |

### EXAMPLE

### PALLAS Study

### I. Patient Selection

Patients with permanent AF were at least 65 years of age with a 12-lead ECG recorded no more than 14 days prior to randomization showing AF or atrial flutter, documentation in the form of ECG rhythm strips or medical reports indicating AF or atrial flutter for at least 6 months prior to randomization without evidence of sinus rhythm in the intervening period. In addition, both the patient and physician must have reached a decision to allow AF to continue without further efforts to restore sinus rhythm.

Patients were randomized to dronedarone 400 mg bid or placebo.

Patients were also required to have at least one of the risk factors for poor vascular outcomes listed in Table 1. The principal exclusion criteria are listed in Table 2.

TABLE 1
Inclusion criteria:

1. Patients in permanent AF defined by the presence of all of the following criteria:

| | |
|---|---|
| a) | Availability of one 12-lead ECG not more than 14 days prior to |
| | randomization, showing AF or atrial flutter |
| b) | Availability of documentation (including either ECG rhythm strips or |
| | medical report of the rhythm) confirming AF or atrial flutter at least 6 |
| | months prior to randomization |
| c) | No evidence of sinus rhythm in the period between these two |
| | documentations of AF |
| d) | Patient and physician decision to allow AF to continue without further |
| | efforts to restore sinus rhythm |

2. Patients aged 65 years or older with at least one of the following risk criteria:

| | |
|---|---|
| a) | Coronary artery disease |
| b) | Prior stroke or TIA (confirmed by a neurologist) |
| c) | Symptomatic heart failure |
| d) | Left ventricular ejection fraction less than or equal to 0.40 measured by |
| | echocardiography within 3 months of randomization |
| e) | Peripheral arterial occlusive disease |
| f) | Aged 75 years or older with both hypertension and diabetes mellitus |

3. Signed written informed consent

TABLE 2
Exclusion criteria
Exclusion criteria related to study methodology

| | |
|---|---|
| 1. | Paroxysmal AF |
| 2. | Persistent AF without a decision to allow AF to continue |
| | without further efforts to restore sinus rhythm |
| 3. | Atrioventricular node ablation or permanent third-degree AV |
| | block, unless patient has a pacemaker |
| 4. | Implanted cardioverter/defibrillator |
| 5. | Any non-cardiovascular illness or disorder that could preclude |
| | participation or severely limit survival, including cancer with |
| | metastasis and organ transplantation requiring immune suppression. |

### Exclusion Criteria Related to the Current Knowledge of Dronedarone

1. NYHA class IV or recent unstable NYHA class III heart failure, where unstable refers to a requirement for intensification of therapy (e.g., increased diuretic, ACE/ARB, or inotropic dosage due to exacerbated symptoms within one month prior to randomization).
2. Sustained daytime bradycardia <50 bpm without a permanent pacemaker
3. QTc interval ≧500 ms, or 530 ms if there is ventricular paced rhythm
4. Hypersensitivity to the active substance or to any of its excipients
5. Need for co-administration of strong CYP3A4 inhibitors, such as ketoconazole, itraconazole, voriconazole, telithromycin, clarithromycin, nefazodone and ritonavir leading to increased systemic exposure to dronedarone
6. Need for co-administration of medicinal products capable of inducing torsades de pointes ventricular tachycardia, such as phenothiazines, cisapride, bepridil, tricyclic antidepressants, terfenadine and certain oral macrolides, or Vaughan Williams class I and III antiarrhythmic agents)
7. Severe hepatic impairment

Any additional contraindication according to local labeling of dronedarone

The term “cardiovascular hospitalization” means a hospitalization which is caused by at least one of the following pathologies:
Acute coronary syndrome
   Myocardial infarction
   Unstable angina pectoris
Transient ischemic attack (TIA) or cerebrovascular event
   Transient ischemic attack
   Ischemic Stroke
   Primary intracerebral hemorrhage (hemorrhagic stroke)
   Ischemic strokes with hemorrhagic transformation
   Primary subarachnoid hemorrhage
Heart failure
Systemic arterial embolism (noncerebrovascular)
Stable angina pectoris
Atrial fibrillation or atrial flutter due to one of the following reasons:
   Symptoms specific to atrial fibrillation or atrial flutter
   Management of anticoagulation
   Management of rate control medication
Bradycardia
Atrio-Ventricular block (complete)
Ventricular arrhythmia or nonfatal cardiac arrest
   Nonfatal cardiac arrest
   Torsades de pointes
   Ventricular fibrillation or out-of-hospital cardiac arrest
   Ventricular tachycardia (nonsustained and sustained ventricular tachycardia)
Syncope
   Loss of consciousness of cardiac or presumed cardiovascular origin
Cardiovascular surgery or intervention
   Cardiovascular surgery
   Implantation of a
      Pacemaker
      ICD
      Other cardiac device
   Transcutaneous procedure
Pulmonary embolism
Deep vein thrombosis
Endocarditis, pericarditis, myocarditis
Other cardiovascular causes

“Cardiac or cardiovascular death” covers, in the context of the invention, mortality due to any cardiovascular causes (any death except those due to a non-cardiovascular cause), in particular classified into arrhythmic, heart failure and other cardiac deaths:
1. Cardiac Arrhythmic Death: refers to death that occurs unexpectedly in a previously stable patient. These deaths are sometimes referred to as sudden deaths and include the following deaths:
   a. Witnessed and instantaneous without new or worsening symptoms
   b. Witnessed and attributed to an identified arrhythmia (e.g., captured on an electrocardiographic (ECG) recording or witnessed on a monitor by either a medic or paramedic)
   c. Subjects unsuccessfully resuscitated from cardiac arrest or successfully resuscitated from cardiac arrest but who die within 24 hours without identification of a noncardiac etiology
   d. Unwitnessed death (information regarding the patient's clinical status within the week preceding death should be provided)
2. Death due to Heart Failure: refers to death occurring in the context of clinically worsening symptoms and/or signs of heart failure without evidence of another cause of death. New or worsening signs and/or symptoms of heart failure (HF) include any of the following:
   a. New or increasing symptoms and/or signs of heart failure requiring the initiation of, or an increase in, treatment directed at heart failure or occurring in a patient already receiving maximal therapy for heart failure
   b. Heart failure symptoms or signs requiring continuous intravenous therapy or oxygen administration
   c. Confinement to bed predominantly due to heart failure symptoms
   d. Pulmonary edema sufficient to cause tachypnea and distress not occurring in the context of an acute myocardial infarction or as the consequence of an arrhythmia occurring in the absence of worsening heart failure
   e. Cardiogenic shock whether or not occurring in the context of an acute myocardial infarction.
This category will not include sudden death occurring during an admission for worsening heart failure unless the patient was expected to die within 3 months.
3. Death due to other cardiac causes: refers to death due to myocardial rupture, pericardial tamponade, valve thrombosis, or as a direct consequence (e.g., bleeding) of a cardiac invasive intervention or surgery.
4. Death due to stroke: refers to death occurring up to 30 days after a suspected stroke based on clinical signs and symptoms as well as neuroimaging and/or autopsy, and where there is no conclusive evidence of another cause of death.
5. Death due to other noncardiac vascular causes: refers to other vascular causes including pulmonary embolism, dissection or rupture of aortic or arterial aneurysm, consequence of a peripheral vascular procedure or invasive intervention within 30 days after the event except if clearly related, systemic arterial embolism
6. Presumed Cardiovascular Death: refers to deaths not attributed to the categories of cardiovascular death and not attributed to a noncardiovascular cause. These are presumed cardiovascular deaths and as such are part of the cardiovascular mortality endpoint.

### II. Duration and Treatment

Treatment was initiated using tablets containing either the placebo or an amount of dronedarone hydrochloride corresponding to 400 mg of dronedarone at a rate of one tablet twice a day with the morning and evening meals.

The anticipated duration of the treatment was variable according to the time at which each patient was included in the study, and could range between 3 months for the last patient included and 3 years for the first patient included.

### III. Results

The results obtained in this trial were analysed by the Kaplan Meier method for the figures, and the relative risk (RR) was estimated using Cox's proportional-effect regression model.

The relative risk (RR) is the ratio of the rates of occurrence of a hospitalization or of a death among the patients on dronedarone, relative to the patients on placebo.

The percentage reduction x of a given event (hospitalization, death, cardiovascular death, etc.) is calculated in the following way:

x=1−relative risk.

III.1. Preliminary Results Relating to Unplanned Cardiovascular Hospitalizations and to Mortality in Patients with Permanent Atrial Fibrillation or Flutter

72 events were recorded in the placebo group, against 114 in the group treated with dronedarone hydrochloride.

The calculated relative risk is 1.62 (95% Cl: 1.20, 2.17) with a p=0.001, i.e. the risk of increase in cardiovascular hospitalizations and deaths is 1.62 on dronedarone hydrochloride.

III.1′. Results Relating to Unplanned Cardiovascular Hospitalizations and to Mortality in Patients with Permanent Atrial Fibrillation or Flutter

67 events were recorded in the placebo group, against 127 in the group treated with dronedarone hydrochloride.

The calculated relative risk is 1.95 (95% Cl: 1.45-2.62-2.17) with a p<0.001, i.e. the risk of increase in cardiovascular hospitalizations and deaths is 1.95 on dronedarone hydrochloride.

III.2. Preliminary Results Relating to Cardiovascular Mortality in Patients with Permanent Atrial Fibrillation or Flutter

6 events were recorded in the placebo group, against 15 in the group treated with dronedarone hydrochloride.

The calculated relative risk is 2.53 (95% Cl: 0.98-6.53) with a p=0.0462, i.e. the risk of increase in cardiovascular deaths is 2.53 on dronedarone hydrochloride.

III.2′. Results Relating to Cardiovascular Mortality in Patients with Permanent Atrial Fibrillation or Flutter

10 events were recorded in the placebo group, against 21 in the group treated with dronedarone hydrochloride.

The calculated relative risk is 2.11 (95% Cl: 1.00-4.49) with a p=0.046, i.e. the risk of increase in cardiovascular deaths is 2.11 on dronedarone hydrochloride.

III.3. Preliminary Results Relating to Unplanned Cardiovascular Hospitalizations in Patients with Permanent Atrial Fibrillation or Flutter

68 events were recorded in the placebo group, against 104 in the group treated with dronedarone hydrochloride.

The calculated relative risk is 1.56 (95% Cl: 1.15-2.12) with a p=0.0039, i.e. the risk of increase in cardiovascular hospitalizations is 1.56 on dronedarone hydrochloride.

III.3′. Results Relating to Unplanned Hospitalizations for Cardiovascular Causes in Patients with Permanent Atrial Fibrillation or Flutter

59 events were recorded in the placebo group, against 113 in the group treated with dronedarone hydrochloride.

The calculated relative risk is 1.97 (95% Cl: 1.44-2.70) with a p<0.001, i.e. the risk of increase in unplanned hospitalizations for cardiovascular causes is 1.56 on dronedarone hydrochloride.

III.4. Preliminary Results Relating to Cardiovascular Hospitalizations for Heart Failure in Patients with Permanent Atrial Fibrillation or Flutter

16 events were recorded in the placebo group, against 35 in the group treated with dronedarone hydrochloride.

The calculated relative risk is 2.21 (95% Cl: 1.22-3.99) with a p=0.0070, i.e. the risk of increase in cardiovascular hospitalizations for heart failure is 2.21 on dronedarone hydrochloride.

III.4′. Results Relating to Cardiovascular Hospitalizations for Heart Failure in Patients with Permanent Atrial Fibrillation or Flutter

24 events were recorded in the placebo group, against 43 in the group treated with dronedarone hydrochloride.

The calculated relative risk is 1.81 (95% Cl: 1.10-2.99) with a p=0.02, i.e. the risk of increase in cardiovascular hospitalizations for heart failure is 1.81 on dronedarone hydrochloride.

III.5. Preliminary Results Relating to Heart Failure in Patients with Permanent Atrial Fibrillation or Flutter

33 events were recorded in the placebo group, against 80 in the group treated with dronedarone hydrochloride.

The calculated relative risk is 2.49 (95% Cl: 1.66-3.74) with a p<0.0001, i.e. the risk of increase of heart failure related episodes is 2.49 on dronedarone hydrochloride.

III.6. Preliminary Results Relating to Cardiovascular Hospitalizations and to Mortality in Patients with Permanent Atrial Fibrillation or Flutter and Symptomatic Heart Failure

The calculated relative risk is 1.46 (95% Cl: 1.01, 2.09) with p=0.043, i.e. the risk of increase of cardiovascular hospitalizations and death is 1.46 on dronedarone hydrochloride.

III.7. Preliminary Results Relating to Cardiovascular Hospitalizations and to Mortality in Patients with Permanent Atrial Fibrillation or Flutter and Left Ventricular Ejection Fraction (LVEF)<40%

The calculated relative risk is 1.66 (95% Cl: 1.20, 2.30) with p=0.002, i.e. the risk of increase of cardiovascular hospitalizations and death is 1.66 on dronedarone hydrochloride.

III.8. Preliminary Results Relating to Cardiovascular Hospitalizations and to Mortality in Patients with Permanent Atrial Fibrillation or Flutter and NYHA Class III Heart Failure

The calculated relative risk is 1.58 (95% Cl: 1.02, 2.44) with p=0.041, the risk of increase of cardiovascular hospitalizations and death is 1.58 on dronedarone hydrochloride.

III.9 Results Relating to a Heart Failure Episode or Hospitalizations for Heart Failure in Patients with Permanent Atrial Fibrillation or Flutter

55 events were recorded in the placebo group, against 115 in the group treated with dronedarone hydrochloride.

The calculated relative risk is 2.16 (95% Cl: 1.57-2.98) with a p<0.001, i.e. the risk of increase in a heart failure episode or hospitalizations for heart failure is 2.16 on dronedarone hydrochloride.

What is claimed is:

1. A method of preventing atrial fibrillation in a patient with a history of paroxysmal or persistent atrial fibrillation or atrial flutter, comprising:
a) initiating dronedarone administration in said patient, and
b) performing electrocardiograms of said patient serially.

2. The method according to claim 1, further comprising:
c) discontinuing dronedarone administration if the patient develops permanent atrial fibrillation or if symptomatic atrial fibrillation reoccurs.

3. The method according to claim 1, further comprising:
c) discontinuing dronedarone administration or cardioverting said patient if atrial fibrillation is detected.

4. The method according to claim 1, further comprising preventing cardiovascular hospitalization.

5. The method according to claim 1, further comprising preventing hospitalization for atrial fibrillation.

6. The method according to claim 1, wherein said prevention of atrial fibrillation is maintenance of sinus rhythm.

7. The method according to claim 1, wherein said patient has non permanent atrial fibrillation or atrial flutter.

8. The method according to claim 1, wherein said patient does not have permanent atrial fibrillation or flutter with additional cardiovascular risk factors.

9. The method according to claim 1, wherein said patient does not have permanent atrial fibrillation nor a history of, or current heart failure.

10. The method according to claim 1, wherein said patient does not have permanent atrial fibrillation nor symptomatic heart failure with recent decompensation requiring hospitalization.

11. The method according to claim 1, wherein said patient does not have permanent atrial fibrillation nor heart failure defined as NYHA class IV.

12. The method according to claim 1, wherein said patient does not have permanent atrial fibrillation nor left ventricular systolic dysfunction.

13. The method according to claim 1, wherein said patient does not have permanent atrial fibrillation, and wherein said permanent atrial fibrillation is defined by one or more of the following:
a) atrial fibrillation duration greater than or equal to 6 months,
b) duration unknown, or
c) attempts to restore sinus rhythm are no longer considered by the physician.

14. The method according to claim 1, wherein said patient does not have permanent atrial fibrillation, and wherein said permanent atrial fibrillation is defined by patients in atrial fibrillation who will not or cannot be cardioverted into normal sinus rhythm.

15. The method according to claim 1, wherein dronedarone or a pharmaceutically acceptable salt thereof is taken twice a day with a meal.

16. A method of risk management of patients developing permanent atrial fibrillation said patients being treated by dronedarone or a pharmaceutically acceptable salt thereof, said method comprising:
a) initiating dronedarone administration in patients with atrial fibrillation or atrial flutter, and
b) performing electrocardiograms of said patient serially.

17. A method of reducing the risk of cardiovascular hospitalization for atrial fibrillation in a patient with a history of paroxysmal or persistent atrial fibrillation comprising:
d) administering dronedarone to said patient, and
e) performing electrocardiograms of said patient serially.

18. The method according to claim 17, further comprising
c) discontinuing dronedarone administration or cardioverting said patient if atrial fibrillation is detected.

19. The method according to claim 17, further comprising preventing hospitalization for atrial fibrillation.

20. The method according to claim 17, wherein said patient does not have permanent atrial fibrillation or flutter with additional cardiovascular risk factors.

21. The method according to claim 17, wherein said patient does not have permanent atrial fibrillation and a history of, or current heart failure.

22. The method according to claim 17, wherein said patient does not have permanent atrial fibrillation and left ventricular systolic dysfunction.

23. The method according to claim 17, wherein the patient does not have permanent atrial fibrillation, and wherein said permanent atrial fibrillation is defined by one or more of the following:
a) atrial fibrillation duration greater than or equal to 6 months,
b) duration unknown, or
c) attempts to restore sinus rhythm no longer considered by the physician.

24. The method according to claim 17, wherein the patient does not have permanent atrial fibrillation, and wherein said permanent atrial fibrillation is defined by patients in atrial fibrillation who will not or cannot be cardioverted into normal sinus rhythm.

25. The method according to claim 17, wherein dronedarone or a pharmaceutically acceptable salt thereof is taken twice a day with a meal.
